(19) United States
(12) Patent Application Publication
(10) Pub. No.: US 2011/0293678 A1
(43) Pub. Date: Dec. 1, 2011

(54) Micellarly integrated oxidation protection for natural dyes

(75) Inventor: Dariush Behnam, Rossdorf (DE)

(73) Assignee: AQUANOVA AG, Darmstadt (DE)

(21) Appl. No.: 13/131,985

(22) PCT Filed: Dec. 1, 2009
(86) PCT No.: PCT/EP2009/008551

§ 371 Date: Aug. 16, 2011

(30) Foreign Application Priority Data
Dec. 1, 2008 (EP) 08020842.4

(51) Int. Cl. A61K 8/11; A23L 1/275; B01J 13/02; A61K 9/50

(52) U.S. Cl. 424/401; 424/490; 426/541; 426/250; 264/4.1

(57) ABSTRACT

In order to formulate natural dyes, which are insoluble or sparingly soluble in water, in such a way that the dye is protected against oxidation, also and in particular after being incorporated into the end product with dilution, and which as a replacement for azo dyes in a beverage result in a solution which is clear to the human eye, the invention provides a solubilizate and a method for preparing a solubilizate. The solubilizate contains at least one natural dye that is insoluble or sparingly soluble in water, at least one emulsifier having an HLB value of 8 to 19, and at least two antioxidants. The dye and the antioxidants are present enclosed together in micelles.

### FIELD OF THE INVENTION

The invention relates to a solubilizate which contains at least one natural dye that is insoluble or sparingly soluble in water, and a method for preparing such a solubilizate.

### BACKGROUND OF THE INVENTION

Formulation of products such as food, cosmetics, and pharmaceuticals in a visually appealing manner is important for positively influencing acceptance by the user. Various substances are available for coloring.

The azo dyes, which include over 2000 different compounds, represent the largest group of dyes. Azo dyes are characterized by their special light fastness, stability, and color intensity, as well as excellent solubility in water. For example, for beverages it is often necessary for the product to be transparent. Azo dyes are best suited for producing clear or transparent beverages.

However, in the human body enzymes break down many azo dyes into their starting compounds, which are highly carcinogenic. Thus, although azo dyes were used in a broad range of applications at the beginning of the 20th century, they are currently used primarily for coloring fats, wood, and paper. Only a few azo dyes are also authorized for coloring foods, in particular transparent beverages, or cosmetic articles and textiles.

Azo dyes are currently suspected of causing allergies and pseudoallergies, as well as being involved in attention deficit hyperactivity disorder. Therefore, in the European Union, for example, in the future foods containing azo dyes will have to display a label stating “May have adverse effects on activity and concentration in children.”

### SUMMARY OF THE INVENTION

In light of this background, the aim of the invention is to allow the use of azo dyes to be dispensed with. Natural dyes represent one option in this regard. In the present context, the term “natural dyes” refers to substances which have a color that is perceivable at least to the human eye and which occur in nature in plants and/or animals. However, replacing the inexpensive azo dyes with natural dyes does not just represent a serious economic problem for the affected industrial sector. Azo dyes in particular are best suited for producing clear or transparent beverages, which heretofore has not been achievable using natural dyes.

Besides the fact that natural dyes are insoluble in water, which essentially precludes use of these dyes in clear beverages, for example, in particular the tendency of these natural substances toward oxidation generally represents a major problem for the user. The original protective function of the natural dyes in plants, for example, is to protect against oxidation and damage from ultraviolet light. Therefore, the natural dyes are extremely sensitive to reactions with oxygen, and are also very sensitive to light. Although it is possible to use isolated natural dyes in the commercial food sector, for example, these natural dyes quickly lose their color intensity due to oxidation processes in the particular foods, such as beverages.

Therefore, these costly dyes must be protected by adding antioxidants. Antioxidants are substances which suppress or retard oxidation processes. Antioxidants have been used for quite some time in the field of food chemistry to prevent spoilage of foods due to oxidation. Ascorbic acid and tocopherols are examples of natural antioxidants.

Currently, antioxidants are added to concentrates or base materials for natural dye products. These concentrates or base materials for natural dye products are then added to the end product in the quantity necessary for achieving the desired coloring of the end product.

After addition, on average, of a few milligrams of dye per kilogram of food, for example a beverage, the volume of the medium containing the dye is increased by a factor of up to 10,000. At the same time, the concentration of the antioxidant originally present in the color concentrate is thus correspondingly diluted by a factor of up to 10,000. At this low concentration, the antioxidant is then available as antioxidation protection for the dyes only to an infinitesimally small, frequently negligible and ineffective degree. As a result, the dyes are oxidized, causing the coloring of the end product to fade.

Conventional natural dye-based formulations for coloring foods, cosmetics, and pharmaceuticals therefore provide the dyes with antioxidation protection only before the formulation is diluted in the end use.

The object of the invention, therefore, is to formulate natural dyes, which are insoluble or sparingly soluble in water, in such a way that the dye is protected against oxidation, also and in particular after being incorporated into the end product, with dilution.

It is an object of the invention in particular to provide a formulation for natural dyes for use in water-based beverages, in which natural dyes are particularly at risk of oxidation from the dissolved oxygen present in the water, wherein the dyes persist essentially completely for the shelf life of the beverage without being oxidized.

These objects are achieved according to the invention using a solubilizate which contains at least one natural dye that is insoluble or sparingly soluble in water, at least one emulsifier having an HLB value of 8 to 19, and at least two antioxidants, the dye and the antioxidants being present enclosed together in micelles. The micelles are formed in particular from a solubilizate which is diluted with water, for example in a ratio of 1 part by weight solubilizate to 1000 parts by weight water.

The solubilizate according to the invention thus allows protection of each antioxidant itself from oxidation, on the one hand by incorporation into the emulsifier shell which forms the micelle, and on the other hand by the chemical oxidation protection provided by the other respective antioxidant, and thus making the antioxidant available, essentially completely and exclusively, for protection of the dye. The invention thus provides for co-solubilization of the dye and the antioxidants. In addition, the dye itself is provided with a diffusion barrier, in the form of the emulsifier shell which forms the micelle, against oxygen and optionally other oxidizing agents which are present in the surroundings of the micelle, in particular in the end use of the dye solubilizate. In this manner, the dye to be protected is provided with optimal antioxidative protection until the expiration date of the end product in question.

Thus, as a result of the invention the antioxidants are provided not in free solution or dissolved in a phase of an emulsion, but, rather, are provided within a micelle, so that they are protected from oxidation in an extremely efficient manner without loss of concentration, even when the solubilizate is incorporated together with the dyes present in the micelle, with dilution.

In principle, hydrophilic and/or lipophilic antioxidants may be used within the scope of the invention. In one preferred embodiment, one of the antioxidants is water-soluble and another antioxidant is fat-soluble. In terms of particularly simple handling, the water-soluble antioxidant may be dissolved in a quantity of water which is just sufficient, and then further processed to form the solubilizate. Another option is to dissolve the water-soluble antioxidant in the at least partially hydrophilic emulsifier, and then carry out further processing to form the solubilizate. According to the invention, when a water-soluble antioxidant and a fat-soluble antioxidant are used, contrary to the professional experience of a chemist skilled in the art the dye, which is insoluble in water but well soluble only in lipophilic solvents, is present within a micelle next to a water-soluble antioxidant, and in a manner not known heretofore may thus be protected from oxidation in an extremely efficient manner.

As explained in detail below with reference to accompanying FIG. 1, due to the configuration of the emulsifier molecules the micelle has defined regions which are formed from lipophilic or hydrophilic molecular sections of the emulsifier molecules. At the same time, the micelle has dimensions in the size range of only a few molecules. According to a model representation, within the scope of the invention the dye molecules and the molecules of the antioxidants are advantageously present in molecularly dispersed form in these regions of the micelle, but consistently in the immediate vicinity of one another in the smallest possible space.

Since the micelle according to the invention is stable enough that it does not open even when the solubilizate is stored over a period of greater than 6 months, preferably greater than one year, particularly preferably at least three years, the oxidation protection does not decrease even when the solubilizate is diluted. According to the invention, the intensity of the natural dyes is thus maintained for at least 6 months, preferably for more than one year, particularly preferably at least three years. The solubilizate is dilutable with water or aqueous media, or also with fats and oils, i.e., hydrophobic media.

Polysorbates, in particular polysorbate 80 and/or polysorbate 20, for example, which are also authorized for foods may be used as emulsifiers.

In addition, either individual sugar esters of edible fatty acids (E 473) or mixtures of at least two sugar esters of edible fatty acids, each in a composition combined with at least one further component which bears at least one OH group, such as water and/or ethanol and/or glycerin, for example, are used as emulsifier, and used in combination with polysorbates or in place of same. Suitable sugar esters of edible fatty acids are in particular sucrose monolaurate, sucrose dilaurate, sucrose monopalmitate, sucrose dipalmitate, sucrose monostearate, and sucrose distearate. At least one medium chain triglyceride (MCT) may also be added to the combination of at least one sugar ester of edible fatty acids and a component which bears at least one OH group, this combination being effective as an emulsifier.

Examples of suitable compositions which act as emulsifiers are listed below, the respective weight proportion being expressed in percent (%):

### Emulsifier Composition A

| Quantity in g | |
|---|---|
| 456 | Distilled water |
| 400 | Sugar esters of edible fatty acids (E473), composed of: |
| | 49.8% sucrose monolaurate |
| | 16.6% sucrose dilaurate |
| | 16.8% sucrose monopalmitate |
| | 16.8% sucrose monostearate |
| 102 | 96% ethanol |
| 42 | Glycerin |

### Emulsifier Composition B

| Quantity in g | |
|---|---|
| 660 | Sugar esters of edible fatty acids (E473), composed of: |
| | 41% sucrose monolaurate |
| | 32% sucrose monopalmitate |
| | 9.0% sucrose dilaurate |
| | 8.5% sucrose dipalmitate |
| | 7.0% sucrose monostearate |
| | 2.5% sucrose distearate |
| 340 | 96% ethanol |

### Emulsifier Composition C

| Quantity in g | |
|---|---|
| 228 | Distilled water |
| 530 | Sugar esters of edible fatty acids (E473), composed of: |
| | 45.13% sucrose monolaurate |
| | 26.93% sucrose monopalmitate |
| | 11.17% sucrose dilaurate |
| | 4.68% sucrose dipalmitate |
| | 10.95% sucrose monostearate |
| | 1.14% sucrose distearate |
| 221 | 96% ethanol |
| 21 | Glycerin |

For preparing the emulsifier compositions, all components are mixed together at room temperature, agitated thoroughly, and homogenized. Assistance may be provided by slightly heating to 48° C. to 52° C., for example.

As antioxidants, ascorbic acid and/or ascorbyl palmitate may advantageously be used as water-soluble antioxidants, and at least one tocopherol and/or carnolic acid and/or BHT and/or BHA and/or TBHQ may advantageously be used as fat-soluble antioxidants.

Ascorbic acid and tocopherol in particular are synergistic in their antioxidative effect, are easy to handle, and in particular are authorized for use in foods. Depending on the field of application, within the scope of the invention the solubilizate may be produced using a-tocopherol and/or β-tocopherol and/or y-tocopherol and/or δ-tocopherol, or using a mixed tocopherol composed of a-tocopherol, β-tocopherol, y-tocopherol, and δ-tocopherol.

It has turned out to be surprising to the inventor and advantageous that the solubilizate does not become turbid, even when diluted with water. The solubilizate is dilutable with water to form a liquid which is transparent, at least to the human eye. This opens up numerous possibilities for using the solubilizate, in particular in beverages, since experience has shown that clear beverages have a particularly high acceptance level with consumers.

Depending on which dyes are to be used for the solubilizate, and optionally, which additional ingredients are to be used for the particular application, the invention provides variation options for the composition, in each case resulting in micelles having sufficient stability. The weight proportions stated below refer to the total mass of the solubilizate. The emulsifier proportion is advantageously in the range of 65% by weight to 90% by weight, preferably in the range of 69% by weight to 85% by weight.

Solubilizates for intensely colored products may be obtained using a dye proportion of less than 20% by weight, preferably in the range of 3% by weight to 18% by weight. However, the invention also allows particularly high loadings of dye greater than 20% by weight, if necessary. Thus, for example, for a loading of 23% by weight of dye, a solubilizate is obtained which, after dissolution in water, contains the dye enclosed in the product micelles and results in a completely clear solution without sedimentation or formation of a fat ring.

The loadings of the solubilizate with dye, which may be achieved by the invention, are thus extraordinarily high compared to commercially available products having loadings generally in the range of 0.4% by weight to approximately 2.5% by weight, in exceptional instances.

Since the antioxidants are protected by the formulation according to the invention in the micelles, a proportion of the water-soluble antioxidant of less than 10% by weight is sufficient, and is preferably in the range of 2% by weight to 5% by weight, particularly preferably 3% by weight. A proportion of less than 5% by weight is sufficient for the fat-soluble antioxidant. The proportion of the fat-soluble antioxidant is preferably less than 3% by weight, and is particularly preferably 1% by weight.

A stable solubilizate for natural dyes is achieved using the formulation according to the invention when the solubilizate is composed of at least one natural dye that is insoluble or sparingly soluble in water, at least one emulsifier having an HLB value of 8 to 19, at least two antioxidants, of which in particular one is water-soluble and the other is fat-soluble, and water.

In one preferred refinement of the invention, the solubilizate contains at least one further fat-soluble adjuvant. It has been shown that the formation of the dye micelles is assisted by adding at least one further fat-soluble adjuvant.

In particular, at least one medium chain triglyceride (MCT) or a mixture of medium chain triglycerides may be used as fat-soluble adjuvant. In the simplest form, a solubilizate having a fat-soluble adjuvant is composed of at least one natural dye that is insoluble or sparingly soluble in water, at least one emulsifier having an HLB value of 8 to 19, at least two antioxidants, of which in particular one is water-soluble and the other is fat-soluble, water, and at least one further fat-soluble adjuvant, in particular a medium chain triglyceride (MCT) or a mixture of medium chain triglycerides.

Under the conditions according to the invention, in particular water-soluble antioxidants such as ascorbic acid, for example, may have a tendency to crystallize out in the micelle with increasing storage time. The stability of the micelles is jeopardized by the crystallizing out of components of the solubilizate, and the micelles may be destroyed, depending on the crystal sizes and crystal structures that form. Crystallizing out may be successfully prevented over a period of at least three years by using a further fat-soluble adjuvant, in particular MCT.

The micelles of the solubilizate, in which the dye and the antioxidant are present enclosed together, have a diameter less than 100 nm, preferably in the range of 4 nm to 30 nm, particularly preferably in the range of 6 nm to 20 nm, measured according to the principle of dynamic light scattering in a 180° backscatter system, using laser light having a wavelength of 780 nm. A liquid which is clear in particular as perceived by the human eye is formed as a result of the small particle sizes. The clarity of the solubilizate may also be characterized by its low turbidity.

For the experimental determination of turbidity, the turbidimeters are calibrated using a standard suspension. Thus, it is the concentration of the calibration suspension, not the measured light intensity, that is displayed. In the measurement of a given suspension, the display thus means that the liquid in question produces the same light scattering as the standard suspension having the indicated concentration. Formazine is the internationally established turbidity standard. The most common unit is the “formazine nephelometric unit” (FNU). This is the unit used in water treatment, for example, for the measurement at 90° according to the specifications of the ISO 7072 standard. The turbidity of the solubilizate according to the invention is less than 30 FNU, preferably less than 20 FNU, and particularly preferably is in the range of 0.5 FNU to 2 FNU, determined by scattered light measurement using infrared light according to the specifications of the ISO 7027 standard, at a 1:1000 dilution of the solubilizate in water.

For preparation of the solubilizate, the invention provides a method which comprises the following steps:
a) Preparing a first mixture of at least one natural dye that is insoluble or sparingly soluble in water, and at least one emulsifier having an HLB value of 8 to 19,
b) Heating the first mixture to a temperature in the range of 80° C. to 150° C., the temperature being adjusted corresponding to the characteristics of the dye,
c) Cooling the first mixture to a temperature in the range of 50° C. to 70° C., preferably in the range of 58° C. to 62° C.,
d) Preparing a second mixture of water and antioxidants,
e) Heating the second mixture to a temperature in the range of 60° C. to 70° C., preferably in the range of 63° C. to 65° C.,
f) Combining the first mixture and the second mixture, with homogenization, and heating to a temperature in the range of 80° C. to 90° C., preferably in the range of 84° C. to 86° C., to form the solubilizate, in which the dye and the antioxidants are present enclosed together in micelles,
g) Cooling the solubilizate to a temperature below 50° C.

In step g) the cooling rate is adjusted as a function of the characteristics of the dye in such a way that the micelles, in which the dye and the antioxidants are present enclosed together, are not destroyed.

For an understanding of the invention, it is important to take into account the findings of the inventor that the micelle formation cannot be achieved solely by preparing a substance mixture in a given mixing ratio. Even by correctly selecting or maintaining the mixing ratio which is the subject matter of the invention, it is possible to obtain only an emulsion in which the dye and the antioxidants are roughly distributed in a dispersion. That is to say, without a uniform structure the emulsion contains different sizes of aggregates or drops of dye and antioxidants in a size range of several microns, i.e., up to one thousand times the size of a micelle.

This antioxidant, which is not micellarly integrated according to the invention, does not provide sufficient protection from oxidation, at the latest in an end product. Such an emulsion is turbid, unlike the micelle-based solubilizate according to the invention, and likewise results in a turbid liquid when diluted with water. The completion of the formation of the micelles specifically loaded with dye and antioxidants according to the invention [in the] production process therefore represents a distinctive feature of the method according to the invention.

For this reason, the completion of the micelle formation in the course of production using the method according to the invention is advantageously monitored with the aid of so-called transmitted light measurement. In one preferred refinement, this monitoring is performed continuously. For this purpose, the preparation of the solubilizate is carried out in an agitator vessel which is provided with a window. A laser light beam is guided through the window and into the agitator vessel. The light passes through the liquid inside the agitator vessel from which the solubilizate is to be prepared, strikes the inner wall of the agitator vessel opposite the window, is reflected at that location, and exits the agitator vessel via the window. Thus, a point of light may be observed on the inner wall of the agitator vessel during preparation of the solubilizate. The micelle formation process is not terminated until the laser beam, aligned perpendicular to the wall of the agitator vessel, is observed as a point of light having a diameter of approximately 5 mm on the opposite inner side of the agitator vessel, with absolutely no light scattering. This may be explained by the fact that the micelles which are formed have a smaller diameter than the wavelength of the visible light of the laser used.

In one advantageous refinement of the method, in addition to the dye and the emulsifier at least one fat-soluble adjuvant, for example a medium chain triglyceride or a mixture of medium chain triglycerides, is used. For this purpose, the method offers various options from which one skilled in the art selects the suitable variant depending on the characteristics, in particular temperature sensitivity, of the dye in question.

The fat-soluble adjuvant may be introduced, for example, in step a) during preparation of the first mixture. In one embodiment of the invention, step a) is carried out as follows:
a1) Mixing the fat-soluble adjuvant, for example a medium chain triglyceride or a mixture of medium chain triglycerides,
a2) Heating the mixture of emulsifier and fat-soluble adjuvant to a temperature in the range of 60° C. to 95° C.,
a3) Combining the dye with the mixture of emulsifier and fat-soluble adjuvant.

Alternatively, step a) may be carried out as follows:
a11) Mixing the dye with at least one fat-soluble adjuvant, for example a medium chain triglyceride or a mixture of medium chain triglycerides,
a22) Heating the mixture of dye and fat-soluble adjuvant to a temperature in the range of 55° C. to 85° C., the temperature being adjusted according to the characteristics of the dye,
a33) Combining the emulsifier with the mixture of dye and fat-soluble adjuvant.

In addition, for preparing the second mixture the method according to the invention provides the option to use at least one fat-soluble adjuvant, for example a medium chain triglyceride or a mixture of medium chain triglycerides, in addition to water and the antioxidants in step d).

The invention further relates to the use of the above-described solubilizate as an additive in foods, in particular beverages, and cosmetics or pharmaceuticals. Since the micelle does not release its content of dye and antioxidants, even in an end product such as a food, in particular a beverage, the antioxidants contained in the micelle are available, essentially exclusively to the dye, as a protective shield, so that the food in question maintains its intense coloration over a long period of time. The invention therefore also relates to a food, in particular a beverage, a cosmetic product, and a pharmaceutical product which contains such a solubilizate.

### BRIEF DESCRIPTION OF THE DRAWINGS

The invention is explained in greater detail below based on exemplary embodiments, with reference to the accompanying figures, which show the following:

FIG. 1 shows schematic illustrations of a model representation for distinguishing an aqueous micellar solution of the solubilizate according to the invention (middle illustration and detailed illustration on the right) from an emulsion (left illustration),

FIG. 2 shows a schematic illustration of the measuring principle of dynamic light scattering for determining the micelle size, based on a diagram at www.particle-metrix.de provided by the manufacturer of the measuring device used,

FIG. 3 shows cumulative number distributions of the solubilizates from samples 1 through 8, measured in an aqueous dilution of 1:1000,

FIG. 4 shows number density distributions of the solubilizates from samples 1 through 8, measured in an aqueous dilution of 1:1000,

FIG. 5 shows measuring results for the particle size distribution for sample 1,

FIG. 6 shows measuring results for the particle size distribution for sample 2,

FIG. 7 shows measuring results for the particle size distribution for sample 3,

FIG. 8 shows measuring results for the particle size distribution for sample 4,

FIG. 9 shows measuring results for the particle size distribution for sample 5,

FIG. 10 shows measuring results for the particle size distribution for sample 6,

FIG. 11 shows measuring results for the particle size distribution for sample 7,

FIG. 12 shows measuring results for the particle size distribution for sample 8, and

FIG. 13 shows a photographic representation of samples 1 through 8, arranged from left to right corresponding to a color gradient from green to yellow to orange.

### DETAILED DESCRIPTION

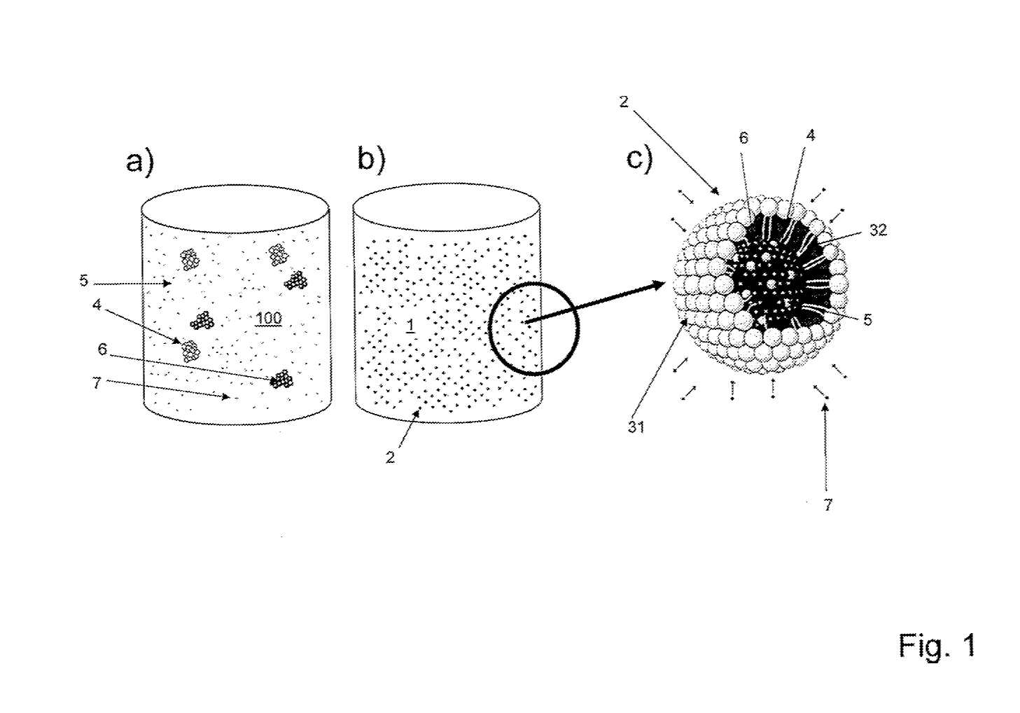

In contrast to the solubilizates according to the invention, conventional products which provide natural dyes in water-dispersible form are formulated as emulsions. The distinction between a solubilizate 1 according to the invention having micelles 2, and an emulsion 100 of a dye 4 is explained in FIG. 1, based on a model representation. Ascorbic acid is used as an example of a hydrophilic antioxidant 5, and tocopherol is used as an example of a hydrophobic antioxidant 6.

In addition to the emulsifier 3, the hydrophilic antioxidant 5 and oxygen 7 are dissolved in the aqueous continuous phase of the emulsion 100. The hydrophobic components of the dye 4 and of the hydrophobic antioxidant 6 are congregated in aggregates or drops. The size of the aggregates or drops is in the range of the wavelength of visible light, so that the emulsion 100 appears turbid to the human eye.

The aggregates or drops of the hydrophobic antioxidant 6 react with the oxygen 7 only at their surface, whereas the antioxidant 6 present inside the aggregate is able to take part in the reaction with oxygen 7, if at all, only very slowly. At the same time, the hydrophilic antioxidant 5, dissolved together with the oxygen 7 in the continuous phase, is consumed very rapidly by the oxidation reaction with the oxygen 7 on account of the molecular distribution. Thus, neither antioxidant 5, 6 adequately protects the dye 4 from oxidation.

In contrast, in an aqueous micellar solution of the solubilizate 1 the hydrophilic antioxidant 5, the hydrophobic antioxidant 6, and the dye 4 are present together in a micelle 2. Molecules of a fat-soluble adjuvant, which when used may likewise be present inside the micelle, are not illustrated for the sake of clarity. In the aqueous phase, oxygen 7 is present outside the micelle. The size of the micelles, having average values around 20 nm, is much smaller than the range of the wavelength of visible light, so that the micellar solution of the solubilizate 1 appears crystal clear to the human eye. The size of the interior of the micelle, in which all components of the solubilizate except for the emulsifier are located, has been estimated to be approximately 5 nm for a solubilizate composed of dye, ascorbic acid dissolved in water, tocopherol, and MCT.

When the solubilizate 1 contacts oxygen 7, only the oxygen 7 which penetrates into the micelle 2 is engaged by the antioxidants 5, 6. As a result of the formulation according to the invention, these antioxidants are able to undergo immediate, complete oxidation upon contact with oxygen. Thus, for the same quantity of antioxidants, the degradation rate of the oxygen is much higher compared to the emulsion 100. At the same time, the dye in the immediate vicinity of the antioxidants is protected, so that pure dye is not attacked by oxygen, as is the case for the emulsion 100.

Since the micelle does not release its contents, even in an end product such as a beverage, the antioxidants contained in the micelle are available exclusively to the dye as a protective shield. Even if an emulsion were prepared which contained the same components, it would not offer the same technological advantage.

Exemplary embodiments for solubilizates of eight different natural dyes are described below. An AGT material number is stated in each case for the ingredients in the formulations. This is a test number, assigned by the present applicant to each substance used, which allows the identity of the respective ingredient used for the solubilizate to be traced. Particle size distribution measurements and turbidity determinations were carried out for some of these solubilizates.

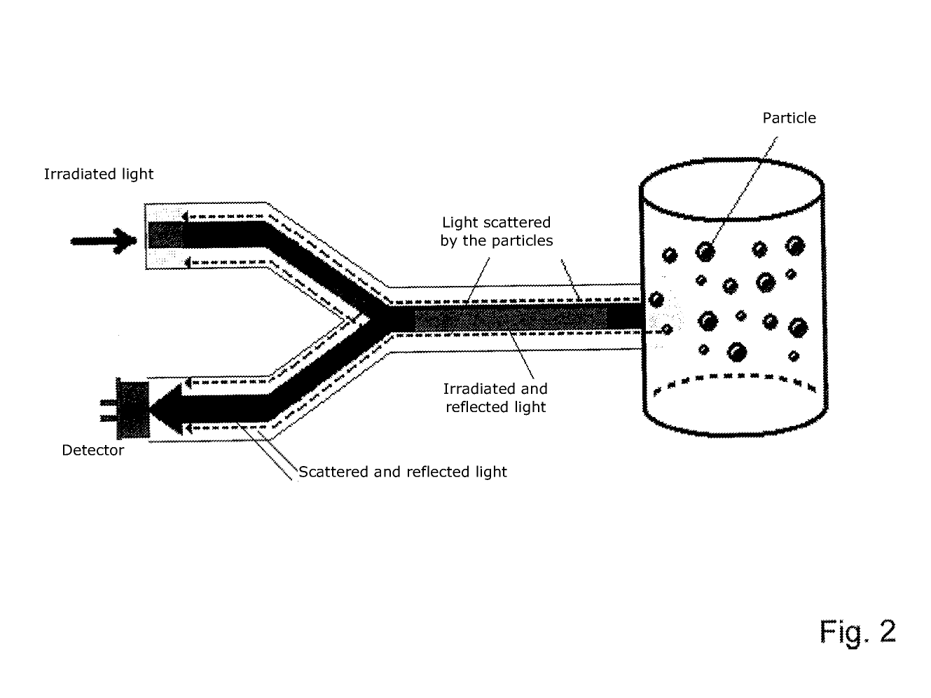

The particle size measurements were performed using the ParticleMetrix Nanotrac backscatter particle analyzer. The measuring principle is based on dynamic light scattering (DLS) in a 180° heterodyne backscatter system. In this geometry, a portion of the laser beam is mixed with the scattered light. This has the same positive effect with regard to the signal-to-noise ratio as the superimposition of all light wavelengths in a Fourier spectrometer. The measuring principle is schematically illustrated in a graphic diagram in FIG. 2.

The laser light having a wavelength of 780 nm is injected on one side of a forked optical fiber. The portion of laser light which is reflected at the glass wall of the measuring container and the backscattered light from the sample return in the same fiber. Both are uniformly distributed in the two branches of the optical fiber. The mixed light is recorded in the detector in the second branch of the forked optical fiber. The fluctuation of the signal resulting from Brownian motion in the scattered light, and thus in the overall signal, is converted to a particle size distribution via the Stokes-Einstein relation and a fast Fourier analysis. The color of the sample has no effect on the quality of the measurement. The detectable particle size range extends from 0.8 nm to 6500 nm. The measurements were carried out in a 1:1000 aqueous dilution. For this purpose, the solubilizate was dissolved in water, with stirring. The solubilizate is completely soluble and clear in water. This solution is stable and transparent.

The turbidity of a sample is based on the fact that incident light striking the suspended particles is scattered by undissolved, finely dispersed substances. The light is scattered in all directions when a linear light beam strikes these particles. Scattered light measurement using infrared light was performed for the turbidity measurement. For small turbidity values, a small quantity of undissolved ingredients may be assumed.

### Exemplary Embodiments

The following exemplary embodiments are based on solubilizates containing ascorbic acid and tocopherol as antioxidants. It is emphasized that within the scope of the invention, other antioxidants may be used instead.

### 1. Solubilizate Containing Zeaxanthin as Dye

The following formulation was used to prepare sample 1:

90 g 20% zeaxanthin FS,
DSM/Brenntag (AGT Material No. 10785/037)

810 g polysorbate 80
Crillet 4, Croda (AGT Material No. 10530/111)

30 g ascorbic acid, BASF (AGT Material No. 10710/005)

30 g distilled water (AGT Material No. 10180)

30 g medium chain triglycerides: Pantex MCT oil,
InCoPa or Delios VK, Cognis (AGT Material No. 10460/102 or 10460/016)

10 g mixed tocopherol, Decanox MTS 70-IP,
ADM/Parmentier (AGT Material No. 10727/001)

The zeaxanthin was heated to a temperature of 84±1° C. The polysorbate 80 was then added as emulsifier, and the mixture was heated to 140±2° C., followed by cooling to 60±2° C. Water, ascorbic acid, MCT oil, and mixed tocopherol were mixed together in a second vessel and heated to 64±1° C., with stirring. The mixture containing zeaxanthin was then added to the mixture containing ascorbic acid/mixed tocopherol. The entire mixture was homogenized and further heated to a temperature of 85±1° C., followed by cooling to a temperature below 50° C. It was then possible to fill the solubilizate.

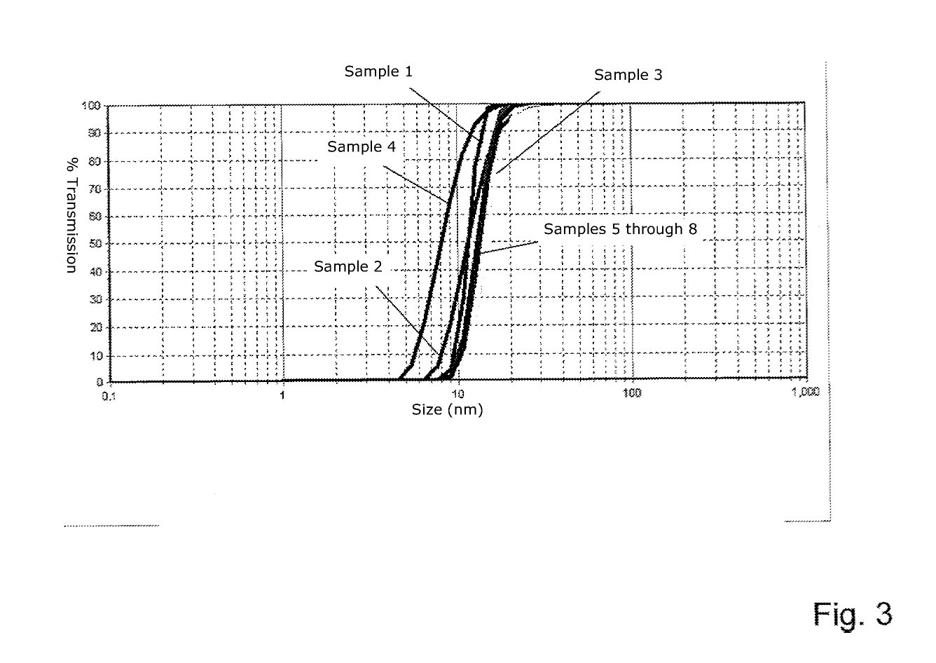

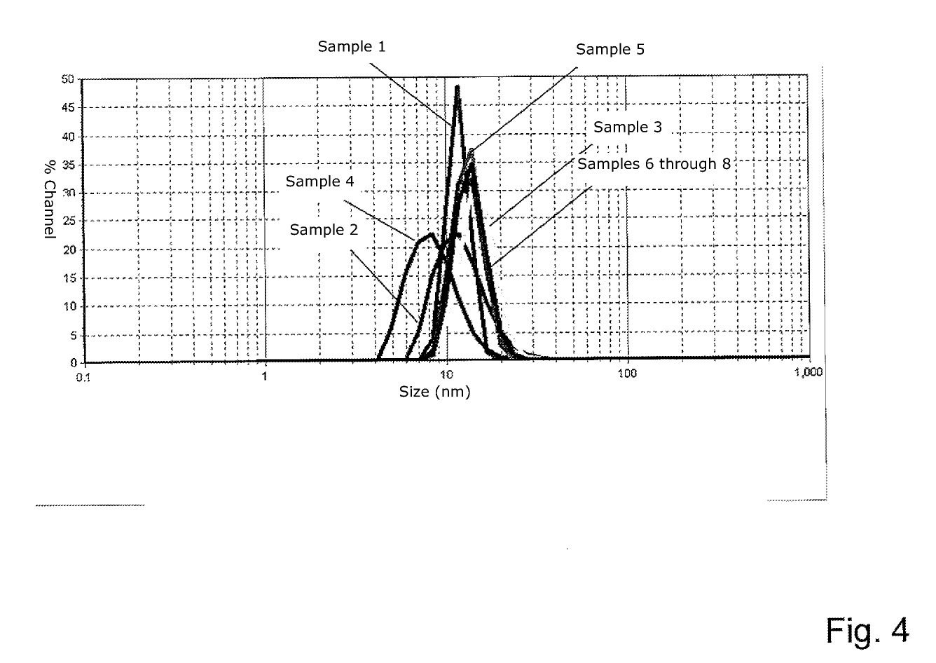

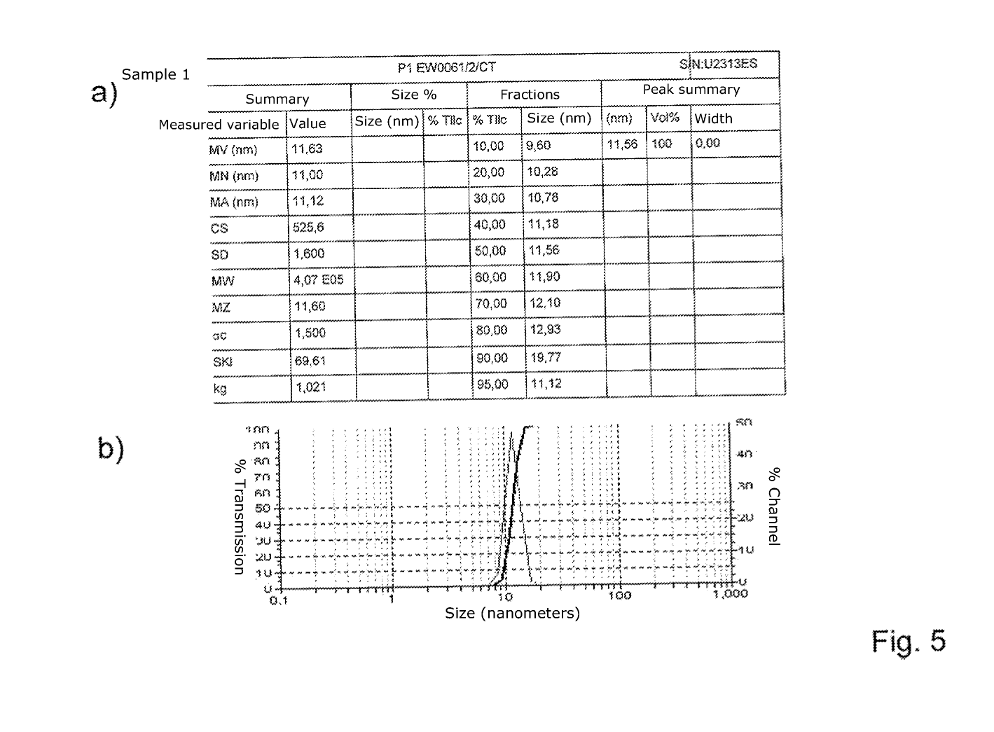

The solubilizate contained 1.5% by weight pure zeaxanthin, and was viscous with a dark red color. Particle size distribution data for the solubilizate are illustrated in FIG. 5. FIGS. 3 and 4 illustrate the cumulative number and number density distributions of the solubilizate of sample 1 compared to the distributions of the other samples.

### 2. Solubilizate Containing Beta-Carotene as Dye

The following formulation was used to prepare sample 2:

64 g beta-carotene: 30% Betatene OLV,
Cognis (AGT Material No. 10091/016)

410 g polysorbate 80, Lamesorb SMO 20, kosher,
Cognis (AGT Material No. 10530/016)

410 g polysorbate 20, Lamesorb SML 20, kosher,
Cognis (AGT Material No. 10520/016)

16 g medium chain triglycerides: Pantex MCT oil,
InCoPa or Delios VK, Cognis (AGT Material No. 10460/102 or 10460/016)

30 g ascorbic acid, BASF (AGT Material No. 10710/005)

30 g distilled water (AGT Material No. 10180)

30 g medium chain triglycerides: Pantex MCT oil,
InCoPa or Delios VK, Cognis (AGT Material No. 10460/102 or 10460/016)

10 g mixed tocopherol, Decanox MTS 70-IP,
ADM/Parmentier (AGT Material No. 10727/001)

The beta-carotene was heated to a temperature of 55±5° C. The polysorbates as an emulsifier mixture and 16 g MCT oil were heated to a temperature of 65±5° C. and mixed. This mixture was added to the beta-carotene, and the resulting mixture was homogenized, with stirring, and further heated to 145±2° C., followed by cooling to 60±2° C. Water, ascorbic acid, 30 g MCT oil, and mixed tocopherol were mixed together in a second vessel and heated to 64±1° C., with stirring. The mixture containing beta-carotene was then added to the mixture containing ascorbic acid/mixed tocopherol. The entire mixture was homogenized and further heated to a temperature of 85±1° C., followed by cooling to a temperature below 50° C. It was then possible to fill the solubilizate.

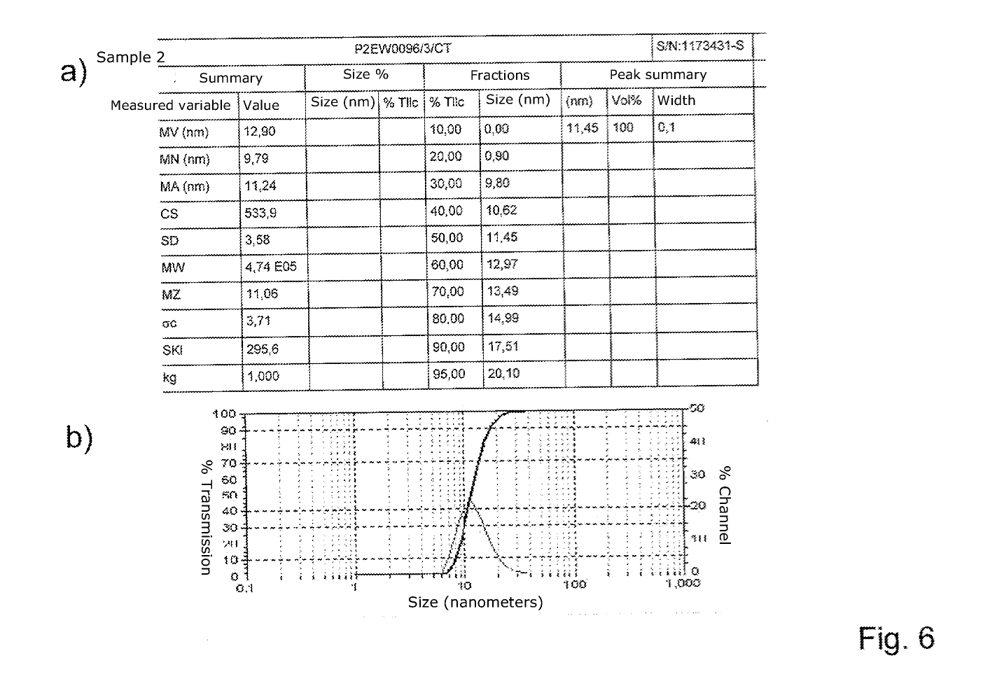

The solubilizate contained 1.9% by weight pure beta-carotene, and was viscous with an intense red color. Particle size distribution data for the solubilizate are illustrated in FIG. 6. FIGS. 3 and 4 illustrate the cumulative number and number density distributions of the solubilizate of sample 2 compared to the distributions of the other samples.

### 3. Solubilizate Containing Paprika Oleoresin as Dye

The following formulation was used to prepare sample 3:

180 g paprika oleoresin, 100,000 CU (E160c),
Henry Lamotte (AGT Material No. 10481/068)

720 g polysorbate 80
Crillet 4, Croda, or Lamesorb SMO 20, Cognis (AGT Material No. 10530/111 or 10530/016)

30 g ascorbic acid, BASF (AGT Material No. 10710/005)

30 g distilled water (AGT Material No. 10180)

30 g medium chain triglycerides, Pantex MCT oil,
InCoPa or Delios VK, kosher, Cognis (AGT Material No. 10460/102 or 10460/016)

10 g mixed tocopherol, Decanox MTS 70-IP,
ADM/Parmentier (AGT Material No. 10727/001)

The paprika oleoresin was heated to a temperature of 60±10° C. The polysorbate was heated to a temperature of 87.5±2.5° C. The paprika oleoresin and polysorbate 80 were then mixed and homogenized at a temperature of 87.5±2.5° C., with stirring, followed by cooling to 60±2° C. Water, ascorbic acid, MCT oil, and mixed tocopherol were mixed together in a second vessel and heated to 64±1° C., with stirring. The mixture containing paprika oleoresin was then added to the mixture containing ascorbic acid/mixed tocopherol. The entire mixture was homogenized and further heated to a temperature of 85±1° C., followed by cooling to a temperature below 50° C. It was then possible to fill the solubilizate.

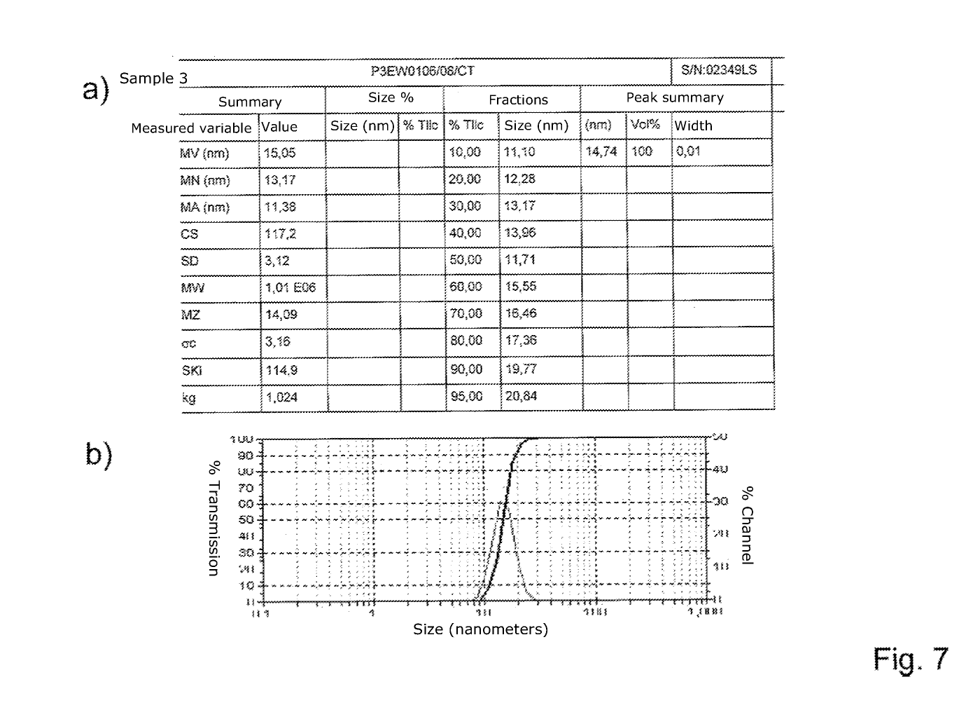

The solubilizate contained 18% by weight paprika oleoresin, and was viscous with an intense red color. Particle size distribution data for the solubilizate are illustrated in FIG. 7. FIGS. 3 and 4 illustrate the cumulative number and number density distributions of the solubilizate of sample 3 compared to the distributions of the other samples.

### 4. Solubilizate Containing Curcumin as Dye

The following formulation was used to prepare sample 4:

60 g curcumin powder,
Akay Flavours & Aromatics containing at least 90% curcumin (AGT Material No. 10175/115)

840 g polysorbate 80,
Crillet 4, Croda (AGT Material No. 10530/111)

30 g ascorbic acid, BASF (AGT Material No. 10710/005)

30 g distilled water (AGT Material No. 10180)

30 g medium chain triglycerides: Pantex MCT oil,
InCoPa or Delios VK, Cognis (AGT Material No. 10460/102 or 10460/016)

10 g mixed tocopherol, Decanox MTS 70-IP,
ADM/Parmentier (AGT Material No. 10727/001)

The polysorbate was heated to a temperature of 50±2° C. The curcumin powder was then added to the polysorbate, with further heating to a temperature of 89±1° C. and with stirring, followed by cooling to 60±2° C. Water, ascorbic acid, MCT oil, and mixed tocopherol were mixed together in a second vessel and heated to 64±1° C., with stirring. The mixture containing curcumin was then added to the mixture containing ascorbic acid/mixed tocopherol. The entire mixture was homogenized and further heated to a temperature of 85±1° C., followed by cooling to a temperature below 50° C. It was then possible to fill the solubilizate.

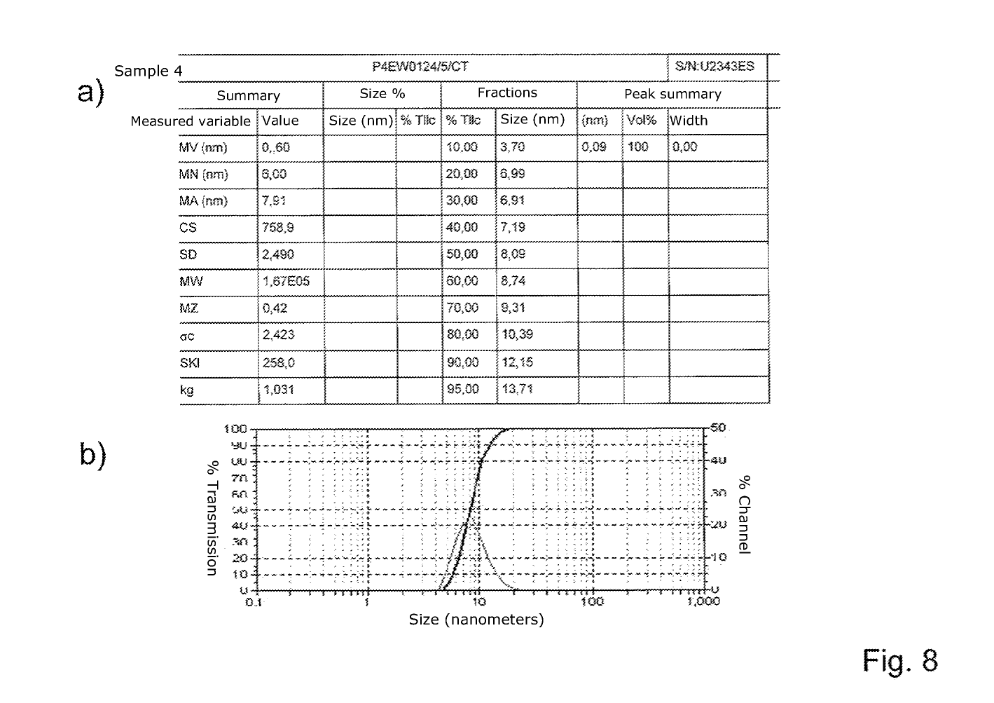

The solubilizate contained 5.5% by weight curcumin, and was viscous with a very dark, intense yellow-orange to reddish color. Particle size distribution data for the solubilizate are illustrated in FIG. 8. FIGS. 3 and 4 illustrate the cumulative number and number density distributions of the solubilizate of sample 4 compared to the distributions of the other samples.

### 5. Solubilizate Containing Apocarotenal as Dye

The following formulation was used to prepare sample 5:

90 g 20% apocarotenal S (20% apocarotenal),
DSM/via Brenntag (AGT Material No. 10093/037)

18 g medium chain triglycerides: Pantex MCT oil,
InCoPa or Delios VK, Cognis (AGT Material No. 10460/102 or 10460/016)

792 g polysorbate 80,
Crillet 4, Croda (AGT Material No. 10530/111)

30 g ascorbic acid, BASF (AGT Material No. 10710/005)

30 g distilled water (AGT Material No. 10180)

30 g medium chain triglycerides: Pantex MCT oil,
InCoPa or Delios VK, Cognis (AGT Material No. 10460/102 or 10460/016)

10 g mixed tocopherol, Decanox MTS 70-IP,
ADM/Parmentier (AGT Material No. 10727/001)

Polysorbate and 18 g MCT oil were heated to a temperature of 65±2° C. and mixed. Apocarotenal was incorporated into this mixture, with stirring, and the resulting mixture was heated to 140±2° C. and homogenized, followed by cooling to 60±2° C. Water, ascorbic acid, MCT oil, and mixed tocopherol were mixed together in a second vessel and heated to 64±1° C., with stirring. The mixture containing apocarotenal was then added to the mixture containing ascorbic acid/mixed tocopherol. The entire mixture was homogenized and further heated to a temperature of 85±1° C., followed by cooling to a temperature below 50° C. It was then possible to fill the solubilizate.

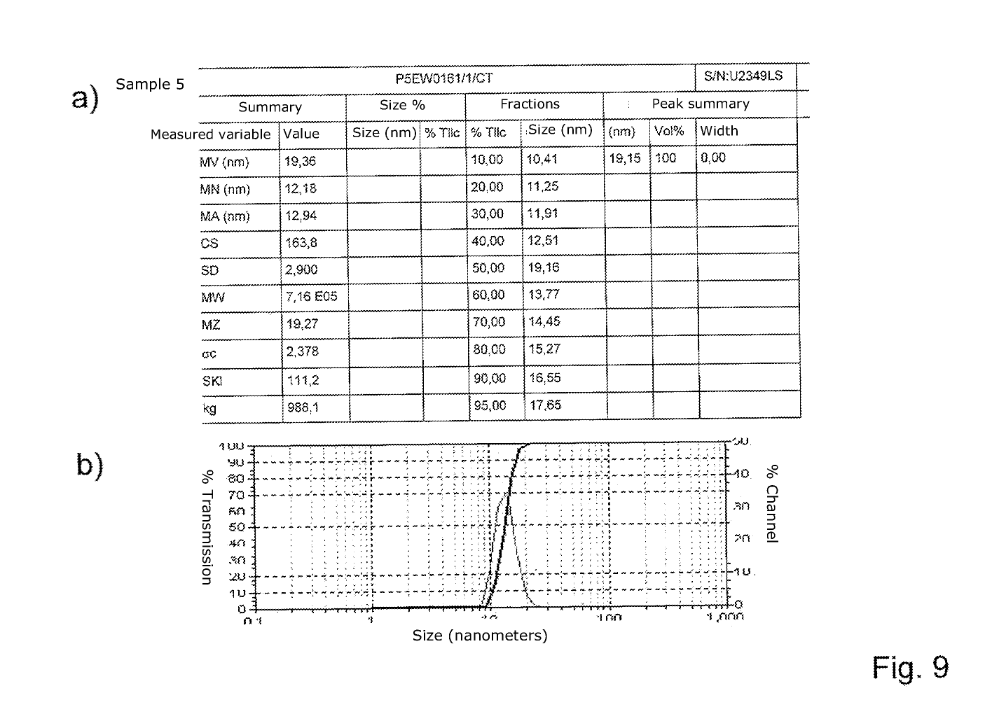

The solubilizate contained 1.5% by weight pure apocarotenal, and was viscous with an intense dark red to brown/black color, yet was clear. Particle size distribution data for the solubilizate are illustrated in FIG. 9. FIGS. 3 and 4 illustrate the cumulative number and number density distributions of the solubilizate of sample 5 compared to the distributions of the other samples.

### 6. Solubilizate Containing Lycopene as Dye

The following formulation was used to prepare sample 6:

35 g lycopene (65%),
CEDA (AGT Material No. 10442/114)

100 g medium chain triglycerides: Pantex MCT oil,
InCoPa or Delios VK, Cognis (AGT Material No. 10460/102 or 10460/016)

765 g polysorbate 80
Crillet 4, Croda (AGT Material No. 10530/111)

30 g ascorbic acid, BASF (AGT Material No. 10710/005)

30 g distilled water (AGT Material No. 10180)

30 g medium chain triglycerides: Pantex MCT oil,
InCoPa or Delios VK, Cognis (AGT Material No. 10460/102 or 10460/016)

10 g mixed tocopherol, Decanox MTS 70-IP,
ADM/Parmentier (AGT Material No. 10727/001)

Lycopene was dissolved in 18 g MCT oil and mixed at a temperature of 60±2° C. Polysorbate was incorporated into this mixture, with stirring, and the resulting mixture was heated to 100±2° C., followed by cooling to 60±2° C. Water, ascorbic acid, MCT oil, and mixed tocopherol were mixed together in a second vessel and heated to 64±1° C., with stirring. The mixture containing apocarotenal was then added to the mixture containing ascorbic acid/mixed tocopherol. The entire mixture was homogenized and further heated to a temperature of 85±1° C., followed by cooling to a temperature below 50° C. It was then possible to fill the solubilizate.

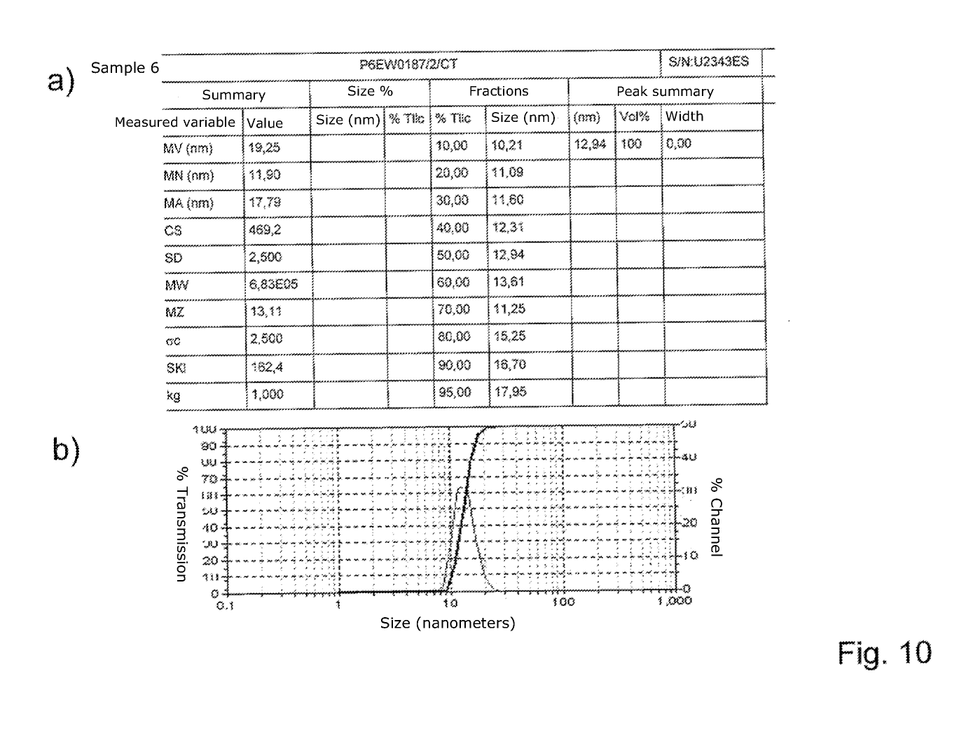

The solubilizate contained 2% by weight pure lycopene, and was viscous with a dark red color. Particle size distribution data for the solubilizate are illustrated in FIG. 10. FIGS. 3 and 4 illustrate the cumulative number and number density distributions of the solubilizate of sample 6 compared to the distributions of the other samples.

### 7. Solubilizate Containing Chlorophyll as Dye

The following formulation was used to prepare sample 7:

60 g chlorophyll 03M K (23%),
Phytone (AGT Material No. 10135/117)

100 g medium chain triglycerides: Pantex MCT oil,
InCoPa or Delios VK, Cognis (AGT Material No. 10460/102 or 10460/016)

740 g polysorbate 80
Crillet 4, Croda (AGT Material No. 10530/111)

30 g ascorbic acid, BASF (AGT Material No. 10710/005)

30 g distilled water (AGT Material No. 10180)

30 g medium chain triglycerides: Pantex MCT oil,
InCoPa or Delios VK, Cognis (AGT Material No. 10460/102 or 10460/016)

10 g mixed tocopherol, Decanox MTS 70-IP,
ADM/Parmentier (AGT Material No. 10727/001)

Chlorophyll was dissolved in 100 g MCT oil and mixed at a temperature of 80±2° C. Polysorbate was incorporated into this mixture, with stirring, and the resulting mixture was heated to 85±1° C., followed by cooling to 60±2° C. Water, ascorbic acid, MCT oil, and mixed tocopherol were mixed together in a second vessel and heated to 64±1° C., with stirring. The mixture containing apocarotenal was then added to the mixture containing ascorbic acid/mixed tocopherol. The entire mixture was homogenized and further heated to a temperature of 85±1° C., followed by cooling to a temperature below 50° C. It was then possible to fill the solubilizate.

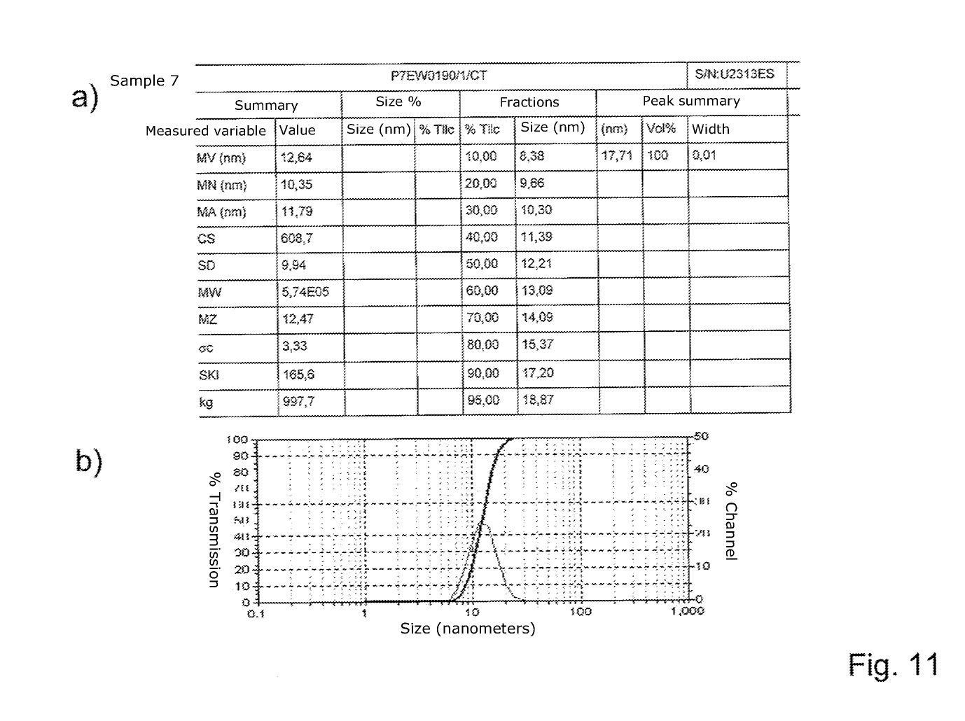

The solubilizate contained 1.2% by weight pure chlorophyll, and was viscous with a dark green/olive color. Particle size distribution data for the solubilizate are illustrated in FIG. 11. FIGS. 3 and 4 illustrate the cumulative number and number density distributions of the solubilizate of sample 7 compared to the distributions of the other samples.

### 8. Solubilizate Containing Lutein as Dye

The following formulation was used to prepare sample 8:

122 g 20% lutein FS,
(DSM) Brenntag (AGT Material No. 10430/114)

18 g medium chain triglycerides: Pantex MCT oil,
InCoPa or Delios VK, Cognis (AGT Material No. 10460/102 or 10460/016)

760 g polysorbate 80,
Crillet 4, Croda (AGT Material No. 10530/111)

30 g ascorbic acid, BASF (AGT Material No. 10710/005)

30 g distilled water (AGT Material No. 10180)

30 g medium chain triglycerides: Pantex MCT oil,
InCoPa or Delios VK, Cognis (AGT Material No. 10460/102 or 10460/016)

10 g mixed tocopherol, Decanox MTS 70-IP,
ADM/Parmentier (AGT Material No. 10727/001)

Polysorbate and 18 g MCT oil were mixed and heated to a temperature of 87±2° C. Lutein was heated and the polysorbate/MCT oil mixture was added thereto, and this mixture was homogenized, with stirring, and further heated to 140±2° C., followed by cooling to 60±2° C. Water, ascorbic acid, MCT oil, and mixed tocopherol were mixed together in a second vessel and heated to 64±1° C. The mixture containing apocarotenal was then added to the mixture containing ascorbic acid/mixed tocopherol. The entire mixture was homogenized and further heated to a temperature of 85±1° C., followed by cooling to a temperature below 50° C. It was then possible to fill the solubilizate.

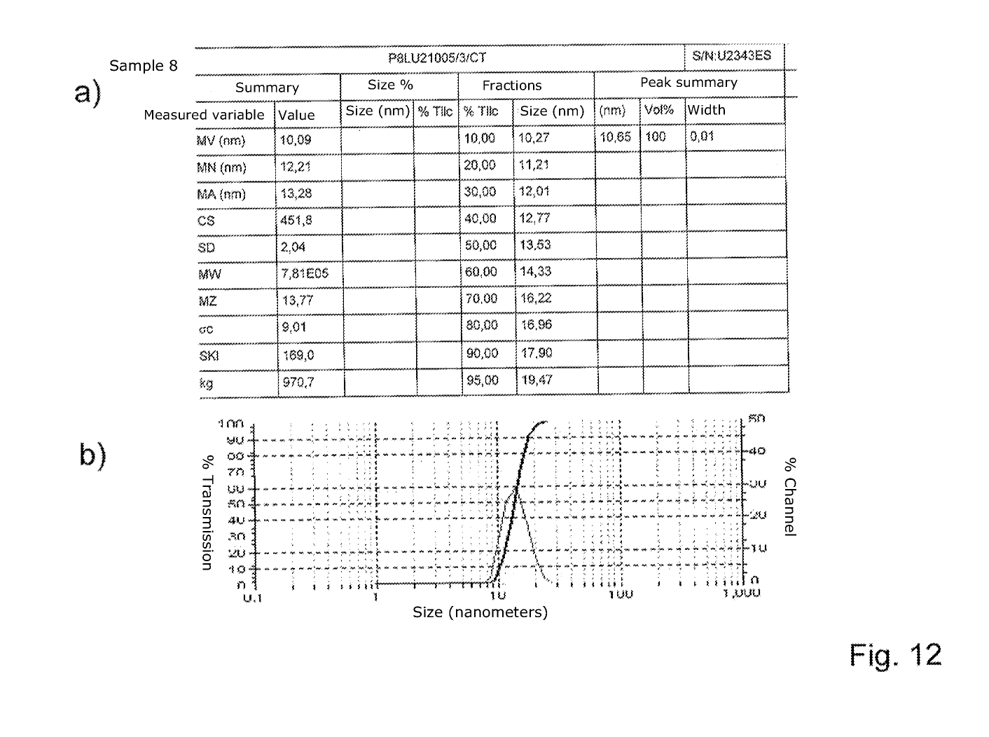

The solubilizate contained 2% by weight pure lutein, and was viscous with an intense dark red to black color. Particle size distribution data for the solubilizate are illustrated in FIG. 12. FIGS. 3 and 4 illustrate the cumulative number and number density distributions of the solubilizate of sample 8 compared to the distributions of the other samples.

As described above, the invention does not depend on the use of water in the solubilizate. For preparing water-free solubilizates, a procedure may be easily carried out as follows, analogously to the exemplary embodiments described above. The use of a water-soluble antioxidant is dispensed with, and instead the proportion of fat-soluble antioxidant is increased. The proportion of emulsifier is also optionally increased. This procedure is demonstrated below, using apocarotenal solubilizate as an example.

### 9. Water-Free Solubilizate Containing Apocarotenal as Dye

The following formulation was used to prepare the water-free solubilizate:

90 g 20% apocarotenal S (20% apocarotenal),
DSM/via Brenntag (AGT Material No. 10093/037)

18 g medium chain triglycerides: Pantex MCT oil,
InCoPa or Delios VK, Cognis (AGT Material No. 10460/102 or 10460/016)

872 g polysorbate 80,
Crillet 4, Croda (AGT Material No. 10530/111)

20 g mixed tocopherol, Decanox MTS 70-IP,
ADM/Parmentier (AGT Material No. 10727/001)

Polysorbate and 18 g MCT oil were heated to a temperature of 65±2° C. and mixed. Apocarotenal was incorporated into this mixture, with stirring, and the resulting mixture was heated to 140±2° C. and homogenized, followed by cooling to 60±2° C. In a second vessel mixed tocopherol was heated to 64±1° C., with stirring. The mixture containing apocarotenal was then added to the mixed tocopherol. The entire mixture was homogenized and further heated to a temperature of 85±1° C., followed by cooling to a temperature below 50° C. It was then possible to fill the solubilizate.

The solubilizate contained 1.5% by weight pure apocarotenal, and was viscous with an intense dark red to brown/black color, yet was clear.

As discussed above, in addition or as an alternative to polysorbate or polysorbates, a mixture of at least one sugar ester of an edible fatty acid (E 473) and at least one further component which bears at least one OH group, such as water and/or ethanol and/or glycerin, for example, may be used as emulsifier in the described examples. The above-referenced examples for emulsifiers A, B, and C provide options for suitable compositions.

One example of 1% apocarotenal solubilizate containing emulsifier A is as follows:

| | | |
|---|---|---|
| 50 g | (5%) | 20% apocarotenal OS, |
| | | Henry Lamotte (AGT Material No. 10093/068) |
| 900 g | (90%) | Emulsifier A, AAG |
| 15 g | (1.5%) | MCT oil Delios V, |
| | | Cognis (AGT Material No. 10460/016) |
| 15 g | (1.5%) | Product water, AAG (AGT Material No. 10180) |
| 15 g | (1.5%) | Ascorbic acid, K & M |
| | | (AGT Material No. 10710/088) |
| 5 g | (1%) | Mixed tocopherol, |

Parmentier (AGT Material No. 10727/038)

MCT oil Delios V, product water, and ascorbic acid were mixed and heated to a temperature of 80-85° C. Emulsifier A was added to this mixture, which was heated to a temperature of 80-85° C. 20% apocarotenal OS was added to this mixture, which was heated to a temperature of 80-85° C. Mixed tocopherol was added to the mixture containing apocarotenal, which likewise was heated to a temperature of 80-85° C., followed by cooling to a temperature below 60° C.

Another example of a 2.4% lutein solubilizate containing emulsifier A is as follows:

| | | |
|---|---|---|
| 30 g | (3%) | 80% lutein powder, |
| | | Henry Lamotte (ATG Material No. 10434/068) |
| 30 g | (3%) | MCT oil Delios V, |
| | | Cognis (ATG Material No. 10460/016) |
| 870 g | (87%) | emulsifier A, AAG |
| 30 g | (3%) | product water, 10180 |
| 30 g | (3%) | ascorbic acid, K & M (ATG Material No. 10710/088) |
| 10 g | (1%) | mixed tocopherol, |
| | | Parmentier (ATG Material No. 10727/038) |

MCT oil Delios V, product water, and ascorbic acid were mixed and heated to a temperature of 80-85° C. In a second vessel emulsifier A was heated to a temperature of 80-85° C., and then 80% lutein powder was added. The mixture containing MCT oil Delios V was then added to this mixture, which was heated to a temperature of 80-85° C. Mixed tocopherol was added to this mixture, which likewise was heated to a temperature of 80-85° C., followed by cooling to a temperature below 60° C.

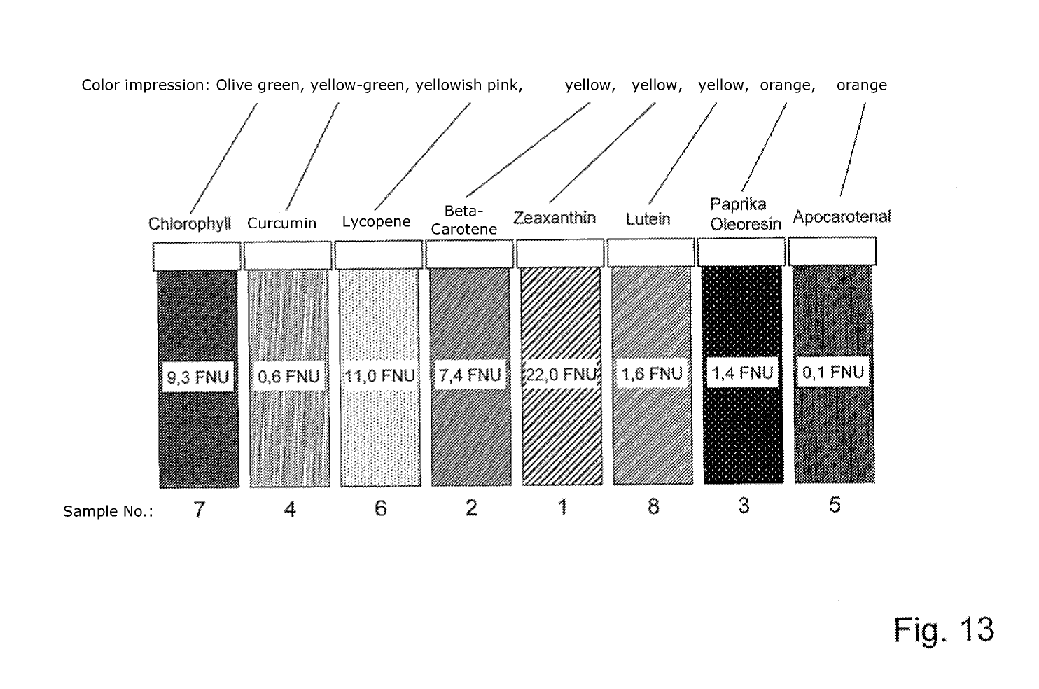

FIG. 13 shows a photographic representation of the solubilizates from samples 1 through 8 in a dilution of 1:1000 in water. The samples are arranged from left to right corresponding to a color gradient from green to yellow to orange. The turbidity measurement values are also stated for each sample. The highest measured turbidity value was 22.0 FNU, which is an extremely low value. This corroborates the visual impression of crystal-clear solutions as observed by the human eye.

Any desired hues between the hues of the individual solubilizates may be produced by mixing the samples with one another. In such a mixing operation according to the invention, the respective concentration of the solubilizates used, as well as their quantity ratio, may be varied in order to obtain the desired hue.

The following statements are based on solubilizates in a 1:1000 dilution with water. A mixture of equal parts curcumin and chlorophyll solubilizates has a hue which is between the respective hue of the chlorophyll solubilizate and the curcumin solubilizate. The hues of the following mixtures are between the hue of the zeaxanthin solubilizate and the hue of the apocarotenal solubilizate which follow the curcumin in the direction toward the apocarotenal. A mixture of equal parts of paprika oleoresin and curcumin has a hue which is slightly redder than the zeaxanthin solubilizate, followed by the hue of a mixture of equal parts of all eight named solubilizates, then a mixture composed of one part apocarotenal and five parts chlorophyll, and lastly, a mixture of equal parts curcumin and apocarotenal.

It is apparent to one skilled in the art that the invention is not limited to the exemplary embodiments described above, and instead may be varied in numerous ways. In particular, the features of the individual exemplary embodiments may also be combined with one another or exchanged with one another.

What is claimed is:

1. A solubilizate containing:
a natural dye that is insoluble or sparingly soluble in water,
an emulsifier having an HLB value of 8 to 19, and
at least two antioxidants,
wherein the natural dye and the at least two antioxidants are present enclosed together in micelles.

2. The solubilizate according to claim 1,
containing at least one water-soluble antioxidant and at least one fat-soluble antioxidant.

3. The solubilizate according to claim 2,
wherein ascorbic acid and/or ascorbyl palmitate form(s) the water-soluble antioxidant and
wherein at least one tocopherol and/or carnolic acid and/or BHT and/or BHA and/or TBHQ form the fat-soluble antioxidant.

4. The solubilizate according to claim 3,
containing a-tocopherol and/or β-tocopherol and/or y-tocopherol and/or δ-tocopherol, or
containing a mixed tocopherol composed of a-tocopherol, β-tocopherol, y-tocopherol, and δ-tocopherol
as tocopherol.

5. The solubilizate according to claim 1,
which is dilutable with water to form a liquid that is transparent to the human eye.

6. The solubilizate according to claim 1,
wherein the emulsifier proportion relative to the total mass of the solubilizate is in the range of 65% by weight to 90% by weight.

7. The solubilizate according to claim 1,
wherein as the emulsifier, polysorbate is used, and/or a composition is used which contains one or more sugar esters of an edible fatty acid (E 473) in combination with at least one component which bears at least one OH group.

8. The solubilizate according to claim 1,
wherein the dye proportion relative to the total mass of the solubilizate is less than 23% by weight.

9. The solubilizate according to claim 1,
wherein the proportion of the water-soluble antioxidant relative to the total mass of the solubilizate is less than 10% by weight.

10. The solubilizate according to claim 1,
wherein the proportion of the water-soluble antioxidant relative to the total mass of the solubilizate is less than 1% by weight.

11. The solubilizate according to claim 1,
wherein the proportion of the fat-soluble antioxidant relative to the total mass of the solubilizate is less than 5% by weight.

12. The solubilizate according to claim 1, composed of:
at least one natural dye that is insoluble or sparingly soluble in water,
at least one emulsifier having an HLB value of 8 to 19, and
at least two antioxidants, of which one is water-soluble and the other is fat-soluble,
and water.

13. The solubilizate according to claim 1,
containing at least one further fat-soluble adjuvant.

14. The solubilizate according to claim 13, composed of:
at least one natural dye that is insoluble or sparingly soluble in water,
at least one emulsifier having an HLB value of 8 to 19, and
at least two antioxidants, of which one is water-soluble and the other is fat-soluble, water, and
at least one further fat-soluble adjuvant.

15. The solubilizate according to claim 1,
wherein the micelles have a diameter in the range of 4 nm to 30 nm, measured according to the principle of dynamic light scattering in a 180° backscatter system, using laser light having a wavelength of 780 nm.

16. The solubilizate according claim 1,
the turbidity of which is less than 30 FNU, determined by scattered light measurement using infrared light according to the specifications of the ISO 7027 standard, at a 1:1000 dilution of the solubilizate in water.

17. A method for preparing a solubilizate, comprising the following steps:
a) preparing a first mixture of at least one natural dye that is insoluble or sparingly soluble in water, and at least one emulsifier having an HLB value of 8 to 19,
b) heating the first mixture to a temperature in the range of 80° C. to 150° C., the temperature being adjusted corresponding to the characteristics of the dye,
c) cooling the first mixture to a temperature in the range of 50° C. to 70° C.,
d) preparing a second mixture of water and antioxidants,
e) heating the second mixture to a temperature in the range of 60° C. to 70° C.
f) combining the first mixture and the second mixture, with homogenization, and heating to a temperature in the range of 80° C. to 90° C. to form the solubilizate, in which the dye and the antioxidants are present enclosed together in micelles, and
g) cooling the solubilizate to a temperature below 50° C.

18. The method according to claim 17,
wherein for preparing the first mixture, in addition to the dye and the emulsifier at least one fat-soluble adjuvant.

19. The method according to claim 18,
wherein step a) is carried out as follows:
a1) mixing the fat-soluble adjuvant,
a2) heating the mixture of emulsifier and fat-soluble adjuvant to a temperature in the range of 60° C. to 95° C., and
a3) combining the dye with the mixture of emulsifier and fat-soluble adjuvant.

20. The method according to claim 18,
wherein step a) is carried out as follows:
a11) mixing the dye with at least one fat-soluble adjuvant,
a22) heating the mixture of dye and fat-soluble adjuvant to a temperature in the range of 55° C. to 85° C., the temperature being adjusted according to the characteristics of the dye, and
a33) combining the emulsifier with the mixture of dye and fat-soluble adjuvant.

21. The method according to claim 17,
wherein for preparing the second mixture, in addition to water and the antioxidants at least one fat-soluble adjuvant.

22. A method comprising:
utilizing the solubilizate according to claim 1 as an additive in foods or cosmetics or pharmaceuticals.

23. Food containing a solubilizate according to claim 1.

24. Cosmetic product containing a solubilizate according to claim 1.

25. Pharmaceutical product containing a solubilizate according to claim 1.
